(19) United States
(12) Patent Application Publication
(10) Pub. No.: US 2023/0065168 A1
(43) Pub. Date: Mar. 2, 2023

(54) VISMODEGIB IN COMBINATION WITH A REPLICATION-DEFICIENT TYPE 5 ADENOVIRUS FOR EXPRESSION OF INTERFERON GAMMA FOR THE TREATMENT OF SKIN CANCER

(71) Applicants: ASCEND BIOPHARMACEUTICALS LTD, South Melbourne (AU); Genentech, Inc., South San Francisco, CA (US)

(72) Inventors: Clement Leong, South Melbourne (AU); Geoffrey Pietersz, South Melbourne (AU); Edward F. McKenna, JR., Calabash, NC (US)

(73) Assignees: ASCEND BIOPHARMACEUTICALS LTD, South Melbourne (AU); Genentech, Inc., South San Francisco, CA (US)

(21) Appl. No.: 17/709,208

(22) Filed: Mar. 30, 2022

Related U.S. Application Data
(60) Provisional application No. 63/169,117, filed on Mar. 31, 2021.

(51) Int. Cl. A61K 31/4418; A61P 35/00; A61P 17/00; C12N 15/86; A61K 38/21

(52) U.S. Cl. CPC A61K 31/4418; A61P 35/00; A61P 17/00; C12N 15/86; A61K 38/217; C12N 2710/10343; C12N 2710/10371

(57) ABSTRACT

Provided are methods of treating skin cancer in an individual in need thereof by administering to the individual vismodegib and a replication-deficient type 5 adenovirus for expression of interferon gamma.

### CROSS-REFERENCE TO RELATED APPLICATIONS

This application claims priority to and benefit of U.S. Provisional Patent Application No. 63/169,117, filed Mar. 31, 2021, and International Patent Application No. PCT/US2021/025269, filed Mar. 31, 2021, the disclosures of which are hereby incorporated herein by reference in their entirety.

### BACKGROUND

The effective treatment of conditions characterized by aberrant cellproliferation, e.g., cancers, remains an enormous challenge to clinicians. Inhibitors of the Hedgehog (Hh) signaling pathway, e.g., vismodegib, have become a valuable addition to the arsenal of agents available for treating cancer and other proliferative disorders. In fact, prior to the introduction of Hh inhibitors, there was no standard therapy for locally advanced or metastatic basal cell carcinoma (BCC). Nevertheless, the extended treatment generally required for treatment with Hh signaling inhibitors, such as vismodegib, and their associated adverse effects on patients, such as muscle spasms, alopecia, dysgeusia, weight loss, fatigue, nausea, diarrhea, decreased appetite, constipation, arthralgias, vomiting, and ageusia, present further disadvantages for the use of Hh signaling inhibitors for treating proliferative disorders. Adverse events associated with administration of vismodegib generally begin to manifest within 6-8 weeks of oral daily treatment.

Surgery is a routine approach in the treatment of skin cancers, but having a non-surgical approach, or at least an approach that can minimize the amount of surgery required, is still an ongoing need, especially in BCC such as BCC involving increasing tumor size, lesions on the central face, poorly defined clinical margins, recurrent BCC such as peri-neural and peri-vascular BCC (which confer higher risk of recurrence). Thus, there is a need for new therapies for the treatment of skin cancers such as BCC which lower the risk of surgery, and also minimize adverse events associated with longer term therapeutic treatment.

### SUMMARY

Provided herein are regimens for the treatment of skin cancer to an individual in need thereof comprising administering vismodegib and a replication-deficient serotype 5 adenovirus for expression of human interferon gamma such as SP-002 (also known as ASN-002).

Various embodiments are contemplated herein. For example, in Embodiment 1, provided is a method of treating skin cancer in an individual in need thereof, comprising:
a) administering to the individual a first treatment cycle of vismodegib and a replication-deficient type 5 adenovirus for expression of interferon gamma, wherein the first treatment cycle comprises oral daily administration of 150 mg vismodegib to the individual for 4 weeks of the first treatment cycle and intralesional injection of the adenovirus to one or more target tumors in a dose range from about 0.5×10¹¹ vp to about 1.5×10¹¹ vp per tumor per dosing day on one day of week 3, one day of week 4, and optionally one day of week 5, of the first treatment cycle;
b) after administering the final dose of the first treatment cycle, providing a drug holiday; and then
c) administering to the individual a second treatment cycle of vismodegib and the adenovirus, wherein the second treatment cycle comprises oral daily administration of 150 mg of vismodegib to the individual for 4 weeks of the second treatment cycle and intralesional injection of the adenovirus to the one or more target tumors in a dose range from about 0.5×10¹¹ vp to about 1.5×10¹¹ vp per tumor per dosing day to the individual one day of week 2, optionally one day of week 3, and optionally one day of week 4, of the second treatment cycle.

Embodiment 2: The method of Embodiment 1,

wherein the first treatment cycle comprises oral daily administration of 150 mg vismodegib to the individual on each one of days 1 to 28 of the first treatment cycle and intralesional injection of the adenovirus to one or more target tumors in a dose range from about 0.5×10¹¹ vp to about 1.5×10¹¹ vp per tumor per dosing day to the individual on day 15, day 22, and optionally day 29 of the first treatment cycle; and

wherein the second treatment cycle comprises oral daily administration of 150 mg of vismodegib to the individual on each one of days 1 to 28 of the second treatment cycle and intralesional injection of the adenovirus to the one or more target tumors in a dose range from about 0.5×10¹¹ vp to about 1.5×10″ vp per tumor per dosing day to the individual on day 8, optionally on day 15, and optionally on day 22, of the second treatment cycle.

Embodiment 3: The method of Embodiment 1, wherein the intralesional injection of the adenovirus to one or more target tumors in a dose range from about 0.5×10¹¹ vp to about 1.5×10¹¹ vp per tumor per dosing day to the individual is given on one day of week 5 or day 29 of the first treatment cycle.

Embodiment 4: The method of any one of Embodiments 1-3, wherein the treatment achieves a beneficial clinical response in at least one of the one or more target tumors after the first treatment cycle, but before the second treatment cycle, wherein the beneficial clinical response is partial response or stable disease.

Embodiment 5: The method of Embodiment 4, wherein the beneficial clinical response is partial response.

Embodiment 6: The method of Embodiment 4, wherein the beneficial clinical response is stable disease.

Embodiment 7: The method of any one of Embodiments 1-6, wherein the treatment does not achieve complete response in at least one of the one or more target tumors after the first treatment cycle, but achieves complete response in at least one of the one or more target tumors after the second treatment cycle.

Embodiment 8: The method of Embodiment 7, wherein the treatment achieves partial response after the first treatment cycle.

Embodiment 9: The method of Embodiment 7, wherein the treatment achieves stable disease after the first treatment cycle.

Embodiment 10: The method of any one of Embodiments 1-3, wherein the treatment achieves clinical response in the one or more target tumors, wherein the clinical response is partial response or stable disease.

Embodiment 11: The method of any one of Embodiments 1-3, wherein the treatment achieves clinical response in at least one of the one or more non-target tumors, wherein the clinical response is partial response or stable disease.

Embodiment 12: The method of any one of Embodiments 1-11, wherein the treatment achieves histological clearance in at least one of the one or more target tumors.

Embodiment 13: The method of any one of Embodiments 1-12, wherein the treatment achieves histological clearance in at least one of the one or more non-target tumors

Embodiment 14: The method of any one of Embodiments 1-3, wherein the treatment achieves complete response and histological clearance in at least one of the one or more target tumors.

Embodiment 15: The method of Embodiment 14, wherein the treatment further achieves complete response and histological clearance in at least one of the one or more non-target tumors.

Embodiment 16: The method of any one of Embodiments 1-15, wherein the drug holiday is at least 8 weeks.

Embodiment 17: The method of any one of Embodiments 1-15, wherein the drug holiday is from about week 5, after the last dose of the first treatment cycle, to and including week 16.

Embodiment 18: The method of Embodiment 17, wherein the second treatment cycle is initiated from about week 17 to about week 20 after the initiation of the first treatment cycle.

Embodiment 19: The method of Embodiment 18, wherein the treatment achieves clinical response in at least one or more target tumors from about week 25 to and including about week 33.

Embodiment 20: The method of any one of Embodiments 1-19, wherein the treatment achieves histological clearance in at least one of the one or more target tumors receiving the adenovirus injection, histological clearance in at least one of the one or more non-target tumors not receiving the adenovirus injection, or both, from about week 25 to and including about week 33.

Embodiment 21: The method of any one of Embodiments 1-20, wherein the treatment achieves a reduction in the rate per year of surgically-eligible BCCs.

Embodiment 22: The method of any one of Embodiments 1-21, wherein the dose of the adenovirus intralesional injection is about 1.0×10¹¹ vp per tumor per dosing day.

Embodiment 23: The method of any one of Embodiments 1-21, wherein the dose of the adenovirus intralesional injection is about 1.5×10¹¹ vp per tumor per dosing day.

Embodiment 24: The method of any one of Embodiments 1-21, wherein the dose of adenovirus intralesional injection is about 0.5×10¹¹ vp per tumor per dosing day.

Embodiment 25: The method of any one of Embodiments 1-24, wherein a volume of the adenovirus intralesional injection is up to about 0.5 mL for tumors with a diameter of no more than 10 mm.

Embodiment 26: The method of any one of Embodiments 1-25, wherein a volume of the adenovirus intralesional injection is up to about 1.0 mL for tumors with a diameter of more than 10 mm.

Embodiment 27: The method of any one of Embodiments 1-26, wherein the skin cancer is selected from the group consisting of basal cell carcinoma (BCC), squamous cell carcinoma, and melanoma.

Embodiment 28: The method of Embodiment 27, wherein skin cancer is basal cell carcinoma (BCC).

Embodiment 29: The method of Embodiment 28, wherein the BCC is Basal Cell Nevus Syndrome, Gorlin syndrome, sporadic BCC, multiple BCC, nodular BCC, superficial BCC, advanced BCC, metastatic BCC, and/or recurrent BCC; and/or the individual has BCC lesions that have recurred after radiotherapy, BCC lesions that are unresectable or surgical resection would result in substantial deformity, BCC involving increasing tumor size, BCC lesions on the central face, BCC with poorly defined clinical margins, recurrent BCC such as peri-neural and peri-vascular BCC, locally advanced BCC that has recurred following surgery and/or in individuals in need thereof who are not candidates for surgery and/or who are not candidates for radiation, or any combination thereof.

Embodiment 30: The method of any one of Embodiments 1-29, wherein the skin cancer comprises more than one target tumor.

Embodiment 31: The method of any one of Embodiments 1-30, wherein the individual is an adult.

### DETAILED DESCRIPTION

Unless specifically defined otherwise, all technical and scientific terms used herein shall be taken to have the same meaning as commonly understood by one of ordinary skill in the art (e.g., in viral vector construction, transfection, gene knockdown, gene knockout, gene therapy, molecular genetics, cancer biology, cancer therapy, immunology, pharmacology, protein chemistry, and biochemistry).

### Definitions

“Treatment”, “treating” or similar phrases refer to obtaining beneficial or desired clinical results for an individual suffering from the skin cancer. Beneficial or desired clinical results includes any one or more of: alleviation of one or more symptoms, diminishment of extent of disease, reducing the number of lesions, abolish all lesions, delaying spread (e.g., metastasis) of disease, delaying recurrence of disease, delay or slowing of disease progression, amelioration of the disease state, and remission (whether partial or total).

“Skin cancer” refers to an abnormal growth of skin cells. Examples of skin cancer include basal cell carcinoma (BCC), squamous cell carcinoma, and melanoma. Examples of BCC include, but is not limited to, Basal Cell Nevus Syndrome, Gorlin syndrome, sporadic BCC, multiple BCC, nodular BCC, superficial BCC, advanced BCC, metastatic BCC, and/or recurrent BCC. In some embodiments. the individual has BCC lesions that have recurred after radiotherapy; the individual has BCC lesions that are unresectable or surgical resection would result in substantial deformity, BCC involving increasing tumor size, BCC lesions on the central face, BCC with poorly defined clinical margins, recurrent BCC such as peri-neural and peri-vascular BCC (which confer higher risk of recurrence), or any combination thereof.

“Tumor size” refers to the largest diameter of palpable tumor that is clinically discernible. For example, BCC tumors (e.g., target or non-target) for clinical study are 5 mm to 20 mm in diameter; surgically-eligible BCC tumors are 5 mm or greater in size.

“Treatment cycle” refers to a period over which a set of doses of vismodegib and the replication-deficient serotype 5 adenovirus for expression of human interferon gamma such as SP-002 (also known as ASN-002), are administered to the individual.

“Individual” refers to a human subject in need thereof suffering from the skin cancer. “Individual”, “subject” and “patient” are used interchangeably herein. The term “adult” refers to an individual 18 years of age or older. In certain embodiments, the individual is a “senior adult”, e.g., 65 years or older. The term “juvenile” refers to an individual 12 to less than 18 years of age.

“Adverse event” refers to any undesirable clinical occurrence in an individual (as compared to the individual's baseline health) and is any untoward medical occurrence defined as an unintended disease or injury or untoward clinical signs (including abnormal laboratory findings) in the individual. More specifically, grades of adverse events, as referred to herein, include those published under the “Common Terminology Criteria for Adverse Events” published by the U.S. National Cancer Institute (version 4.03 published 14 Jun. 2010). These include mild (grade 1) adverse events which present as mild symptoms not requiring medical intervention; moderate (grade 2) adverse events, which require minimal, local or noninvasive intervention; severe or medically significant (grade 3) adverse events, which are not immediately life-threatening, but may require hospitalization or prolongation of hospitalization; life-threatening (grade 4) adverse events requiring urgent intervention; and adverse event-related death (grade 5).

“Clinical response” is a change in tumor size based on the following criteria for clinical response: 1) complete response (CR) is an absence of detectable disease; 2) partial response (PR) is ≥30% decrease in maximum diameter of target tumor; 3) stable disease is any change that did not meet criteria for complete response, partial response, or progressive disease; and 4) progressive disease (PD) is an increase of ≥20% in the maximum tumor diameter. A beneficial clinical response reflects an improved response upon treatment (e.g., CR, PR, or stable disease). The clinical response evaluation tool, as shown in Table 1. The largest diameter of palpable tumor that is clinically discernible is defined as the tumor size.

TABLE 1
Clinical Response Evaluation Tool

| | |
|---|---|
| Palpable Tumor mass: | Yes ( ) |
| | Indeterminate* ( ) No ( ) |
| If a definitive palpable tumor | Diameter of Palpable Thickness ( ) mm |
| is clinically discernible: | |

*Sufficient residual oedema or induration, resulting in uncertainty regarding presence of tumor.

“Histological clearance” refers to the absence of detectable evidence of tumor cell nests in serial histological samples as determined by central pathology review.

“Vismodegib” is a Hedgehog (Hh) pathway inhibitor, which is described chemically as 2-Chloro-N-(4-chloro-3-(pyridin-2-yl)phenyl)-4-(methylsulfonyl)benzamide having the structural formula:

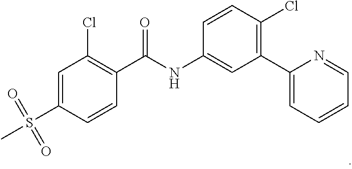 [SMILES: CS(=O)(=O)c1ccc(C(=O)Nc2ccc(Cl)c(-c3ccccn3)c2)c(Cl)c1]

Vismodegib is disclosed in international patent application WO2006028958 titled “Pyridyl Inhibitors of Hedgehog Signaling”. Descriptions of vismodegib and methods of making vismodegib can be found in, e.g., compound 36 and Example 37 of the above referenced application. Vismodegib is also disclosed in Robarge et al (2009) Bioog Med Chem Lett 19, 5576 titled “GDC-0449—A potent inhibitor of the hedgehog pathway”, as compound 31 (GDC-0449); see, e.g., Table 2, Table 3, FIG. 2, and page 5580 column II. Vismodegib is commercially available in 150 mg capsules for oral use as the crystalline free base, sold as Erivedge®. Vismodegib is indicated for the treatment of adults with metastatic basal cell carcinoma, or with locally advanced basal cell carcinoma that has recurred following surgery or who are not candidates for surgery and who are not candidates for radiation. Adverse events commonly associated with administration of vismodegib to patients with advanced BCC include, but are not limited to, muscle spasms, alopecia, dysgeusia, weight loss, fatigue, nausea, diarrhea, decreased appetite, constipation, arthralgia, vomiting, and ageusia. Adverse events associated with administration of vismodegib generally begin to manifest within 6-8 weeks of oral daily treatment.

“SP-002”, also known as ASN-002 or TG1042, refers to a replication-deficient type 5 adenovirus for expression of human interferon gamma; see, e.g., Urosevic (2007) Curr Opin Investig Drugs 8:493-498; Liu et al. (2004) PNAS USA 101 Suppl 2:14567-14571; Dummer et al. (2004) Blood. 104:1631-1638; Dummer et al. (2010) Mol Ther. 18:1244-1247; Accart et al. (2013) J Transl Med. 11:226; Khammari et al. (2015) Cancer Immunol Immunother 64:805-815; Dreno et al. (2014) PLoS One 9:e83670; and Hillman et al. (2004) Cancer Gene Therapy 11:61-72. SP-002 (ASN-002) is a biological intervention/treatment. SP-002 (ASN-002) has been designed for clinical applications, especially for intratumoral administration in the treatment of various cancers. Recombinant adenovirus (rAd) vector delivers the gene of interest, in the case of SP-002 (ASN-002) the human interferon gamma (IFNγ) gene, into target cells. The recombinant adenovirus (rAd) vector in SP-002 (ASN-002) is replication deficient and although it infects cells, it is not able to replicate in the tumor or in normal human cells. The infected cells are able to transcribe and translate the IFNγ DNA leading to a sustained local concentration of IFNγ in the tumor mass that is designed to avoid high levels of systemic IFNγ that may lead to unacceptable toxicity. An extended release formulation of SP-002 (ASN-002) is disclosed in U.S. patent application US20210038660A1 titled “Enhanced Viral Delivery Formulation”; see, e.g., Example 1 “Preparation and In Vitro Testing of Injectable SP-002 (ASN-002) Adenovirus Vector Silica Hydrogel Depot Formulations” in the above referenced application. SP-002 (ASN-002) combination therapies for the treatment of cancer are also disclosed in U.S. patent application US 2019/0269711. The most frequently observed adverse events associated with administration of SP-002 (ASN-002) in adult patients with low-risk nodular BCC include, but are not limited to, injection site reactions (erythema, induration, ulceration, and pain) and flu like symptoms (headache, malaises, and fever).

Reference to “about” a value or parameter herein includes (and describes) variations that are directed to that value or parameter per se. In some embodiments, the term “about” refers to +/−10%, +/−5%, or +/−1%, of the designated value.

The singular forms “a,” “or,” and “the” include plural referents unless the context clearly dictates otherwise.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

### Methods

Described herein are methods of treating skin cancer in an individual in need thereof with a combination of vismodegib and a replication-deficient type 5 adenovirus for expression of interferon gamma (e.g., SP-002 (ASN-002)).

For example, in some embodiments, provided is a method of treating skin cancer in an individual in need thereof, comprising administering to the individual a first treatment cycle of vismodegib and a replication-deficient type 5 adenovirus for expression of interferon gamma (e.g., SP-002), wherein the first treatment cycle comprises oral daily administration of 150 mg vismodegib to the individual for 4 weeks of the first treatment cycle and intralesional injection of the adenovirus to one or more target tumors in a dose range from about 0.5×10” vp to about 1.5×10″ vp per tumor per dosing day on one day of week 3, one day of week 4, and optionally one day of week 5, of the first treatment cycle.

In certain embodiments, the method further comprises administering to the individual a second treatment cycle of vismodegib and the adenovirus, wherein the second treatment cycle comprises oral daily administration of 150 mg of vismodegib to the individual for 4 weeks of the second treatment cycle and intralesional injection of the adenovirus to the one or more target tumors in a dose range from about 0.5×10¹¹ vp to about 1.5×10¹¹ vp per tumor per dosing day to the individual one day of week 2, optionally one day of week 3, and optionally one day of week 4, of the second treatment cycle.

In certain embodiments, the method further comprises a drug holiday between treatment cycles. The term “drug holiday” as used herein, refers to a period over which no drug is administered to the individual. In certain embodiments, the drug holiday is at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 12 weeks, at least about 1 month, at least about 2 months, at least about 2.5 months, or at least about 3 months. In certain embodiments, the drug holiday is about 4 weeks (e.g., at least 1 month) up to and including about 12 weeks (e.g., at least 3 months). Thus, in certain embodiments, there is a drug holiday after injection of the final optional dose (on week 5) of the first treatment cycle, and continues until the first dose of the second treatment cycle. For example, in certain embodiments, there is a drug holiday after injection of the final dose (e.g., the optional dose on week 5) of the first treatment cycle, and continues until the first dose of the second treatment cycle.

In certain embodiments, the second treatment cycle is not required, and the treatment ends after the first treatment cycle. Alternatively, in some embodiments, the second treatment cycle is required. For example, for individuals who do not achieve treatment after a first treatment cycle, or for individuals who may benefit from more than one treatment cycle, a second treatment cycle may provide effective treatment. In some embodiments, an individual who receives a second treatment cycle is an individual who does not achieve a complete response (e.g., only achieves partial response or stable disease, or has progressive disease) after a first treatment cycle. Thus, also provided is a method of treating skin cancer in an individual in need thereof, comprising a) administering to the individual a first treatment cycle of vismodegib and a replication-deficient type 5 adenovirus for expression of interferon gamma (e.g., SP-002), wherein the first treatment cycle comprises oral daily administration of 150 mg vismodegib to the individual for 4 weeks of the first treatment cycle and intralesional injection of the adenovirus to one or more target tumors in a dose range from about 0.5×10¹¹ vp to about 1.5×10¹¹ vp per tumor per dosing day on one day of week 3, one day of week 4, and optionally one day of week 5, of the first treatment cycle; b) after administering the final dose of the first treatment cycle, providing a drug holiday; and then c) administering to the individual a second treatment cycle of vismodegib and the adenovirus, wherein the second treatment cycle comprises oral daily administration of 150 mg of vismodegib to the individual for 4 weeks of the second treatment cycle and intralesional injection of the adenovirus to the one or more target tumors in a dose range from about 0.5×10¹¹ vp to about 1.5×10¹¹ vp per tumor per dosing day to the individual one day of week 2, and optionally one day of week 3 and optionally one day of week 4, of the second treatment cycle.

“Week X” refers to a specified week (e.g., 7 consecutive days) in a treatment cycle or completed treatment. For example, the first treatment cycle is initiated at week 1. In some embodiments a first treatment cycle is from week 1 to about week 4. In some embodiments a first treatment cycle is from week 1 to about week 5. In some embodiments the second treatment cycle is initiated, e.g., from week 9 to and including week 12, from week 13 to and including week 16, from week 14 to and including week 17, from week 15 to and including week 18, from week 16 to and including week 19, or from week 17 to and including week 20, after the initiation of the first treatment cycle. In some embodiments a second treatment cycle is initiated at about week 17. In some embodiments a second treatment cycle is from about week 17 to and including about week 20. In some embodiments, there is (i) a first treatment cycle, e.g., from week 1 to and including week 4 or from week 1 to and including the day of the final optional injection of the adenovirus in week 5; (ii) a drug holiday, e.g., from week 5 (after the final optional injection of the adenovirus); and (iii) a second treatment cycle, e.g., from about week 17 to and including about week 20.

“Day X” refers to a specified day in a treatment cycle. For example, day 1 of the first treatment cycle is the first day of the first treatment cycle and day 1 of the second treatment cycle is the first day of the second treatment cycle. It should be understood that there is some flexibility with the “day” of administration, e.g., for example, “day” may include plus or minus 2 days on either side of the given day assigned for treatment, especially for the day assigned for the individual to receive injection of the adenovirus (e.g., SP-002 (ASN-002)). Adenovirus administration may also be deferred by a week in any tumor as determined by a physician, e.g., for example, if at the target tumor to be treated there is an inflammatory response of Grade 3. The duration of a treatment cycle can be measured by days, weeks, or a combination thereof.

In some embodiments the first treatment cycle comprises oral daily administration of 150 mg vismodegib to the individual on each one of days 1 to 28 of the first treatment cycle and intralesional injection of the adenovirus to one or more target tumors in a dose range from about 0.5×10″ vp to about 1.5×10″ vp per tumor per dosing day to the individual on day 15, day 22, and optionally day 29 of the first treatment cycle. In certain embodiments, the day 29 injection is not required, and the first treatment cycle comprises oral daily administration of 150 mg vismodegib to the individual on each one of days 1 to 28 of the first treatment cycle and intralesional injection of the adenovirus to one or more target tumors in a dose range from about 0.5×10¹¹ vp to about 1.5×10¹¹ vp per tumor per dosing day to the individual on day 15 and day 22. Alternatively, the day 29 injection is required, and, in some embodiments the intralesional injection of the adenovirus to one or more target tumors in a dose range from about 0.5×10¹¹ vp to about 1.5×10¹¹ vp per tumor per dosing day to the individual is given on one day of week 5 or day 29 of the first treatment cycle.

In some embodiments, the second treatment cycle comprises oral daily administration of 150 mg of vismodegib to the individual on each one of days 1 to 28 of the second treatment cycle and intralesional injection of the adenovirus to the one or more target tumors in a dose range from about 0.5×10¹¹ vp to about 1.5×10¹¹ vp per tumor per dosing day to the individual on day 8, and optionally day 15, and optionally day 22, of the second treatment cycle.

The number of treatment cycles an individual receives may range from 1 to 4, e.g., a first treatment cycle, a second treatment cycle, a third treatment cycle, and a fourth treatment cycle. In some embodiments the treatment includes at least a first treatment cycle (e.g., from week 1 to and including week 4 or from week 1 to and including the final optional dose of the adenovirus in week 5) and a second treatment cycle (e.g., from week 9 to and including week 12, from week 13 to and including week 16, from week 14 to and including week 17, from week 15 to and including week 18, from week 16 to and including week 19, or from week 17 to and including week 20). If more than two treatment cycles, e.g., if a third and/or fourth treatment cycle is desired, e.g., as determined by a physician, in certain embodiments, the second treatment cycle regimen is repeated for the additional third and/or fourth treatment cycles. Where an individual is treated over more than one treatment cycle, the total aggregate dose of vismodegib and/or the adenovirus (e.g., SP-002 (ASN-002)) may be varied among the second, third and fourth treatment cycles. Alternatively, in certain embodiments, the dose is the same among the second, third and fourth treatment cycles. Intralesional injection of the adenovirus (e.g., SP-002 (ASN-002)) may be administered to the one or more target tumors to the individual on two or three separate days (e.g., one day of week 3 or day 15, one day of week 4 or day 22, and optionally one day of week 5 or day 29) of the first treatment cycle and on one, two or three single days (e.g., one day of week 2 or day 8, and optionally one day of week 3 or day 15 and/or optionally one day of week 4 or day 22) of the second, third, and fourth treatment cycles. Where an individual is treated over more than one treatment cycles, e.g., a second, third and/or fourth treatment cycle, there is a drug holiday between each treatment cycle. In some embodiments, in conjuction with embodiments above or below, a target tumor is a tumor to which SP-002 is administered by intralesional injection. In some embodiments, in conjuction with embodiments above or below, a non-target tumor is a tumor in which no SP-002 is administered by intralesional injection, but still demonstrates a clinical response and/or histological clearance to the treatment regimen.

In some embodiments, an intralesional injection of the adenovirus (e.g., SP-002 (ASN-002)) is administered to one or more target tumors (e.g. 1, 2, 3, 4, 5, 6, 7, 8, 9, 10, or more target tumors) per dosing day. In some embodiments a dose range of the intralesional injection of the adenovirus (e.g., SP-002 (ASN-002)) to the one or more target tumors is from about 0.5×10¹¹ viral particles (vp) to about 1.5×10¹¹ vp per tumor per dosing day. For example, the dose of the intralesional injection of the adenovirus (e.g., SP-002 (ASN-002)) to the one or more target tumors is 0.5×10¹¹ vp per tumor per dosing day, 1.0×10″ vp per tumor per dosing day, or 1.5×10¹¹ vp per tumor per dosing day. The volume of the adenovirus (e.g., SP-002 (ASN-002)) intralesional injection may be dependent on the tumor size. In embodiments, the volume of the adenovirus (e.g., SP-002 (ASN-002)) intralesional injection is up to about 0.5 mL (e.g., about 0.25 mL to and including about 0.5 mL) for tumors less than 10 mm in diameter and up to about 1.0 mL (e.g., about 0.5 mL to and including about 1.0 mL) for tumors greater than 10 mm in diameter. For example, the volume of the adenovirus (e.g., SP-002 (ASN-002)) intralesional injection is up to about 0.5 mL for tumors about 5 to about 10 mm in diameter and up to about 1.0 mL for tumors about 11 to about 20 mm in diameter. In certain embodiments, the volume of the adenovirus (e.g., SP-002 (ASN-002)) intralesional injection is up to about 0.5 mL for tumors about 5 to about 10 mm in diameter. In certain embodiments, the volume of the adenovirus (e.g., SP-002 (ASN-002)) intralesional injection is up to about 1.0 mL for tumors about 11 mm to about 20 mm in diameter.

In some embodiments, the methods comprise determining whether an individual who received a first treatment cycle is in need of a second (and optionally third and/or fourth) treatment cycle. Eligibility of an individual for a second (and optionally third and/or fourth) treatment cycle may be determined by a treating physician using the clinical response evaluation tool, histological clearance evaluation, and/or at physician's discretion.

To determine the eligibility of an individual for more than one treatment cycle (e.g. two, three, or four treatment cycles), clinical response may be evaluated after the first treatment cycle (e.g. during the drug holiday). Clinical response may include complete response, partial response, stable disease, or progressive disease. Clinical response may be evaluated on target tumors, non-target tumors, or both. An individual who achieves partial response, stable disease, or progressive disease after the completion of the first or subsequent treatment cycle may, at treating physician's discretion, continue to receive the next treatment cycle. An individual who does not achieve complete response (e.g., but who has achieved partial response or stable disease, or has progressive disease) after the completion of the first treatment cycle may achieve complete response after the second, third, or fourth treatment cycle.

Clinical response may be assessed at any suitable time or times during or after the disclosed treatment. In some embodiments, clinical response is determined after the completion of a first treatment cycle. In some embodiments, clinical response is determined after the completion of the second, third, and/or fourth treatment cycle. In some embodiments, clinical response is determined after the completion of the first and second (and subsequent) treatment cycles. In some embodiments, clinical response is determined after the completion of a first treatment cycle but before the initiation of a second treatment cycle. In some embodiments, clinical response is determined at about week 9, about week 10, about week 11, about week 12, about week 13, about week 14, about week 15, about week 16, about week 17, or about week 18, e.g., prior to or at the start of or during the second treatment cycle. In some embodiments, clinical response is determined after the completion of the second treatment cycle. In some embodiments, clinical response is determined from about week 25 to and including about week 33, e.g., about week 25, about week 26, about week 27, about week 29, about week 29, about week 30, about week 31, about week 32, or about week 33. In some embodiments, clinical response is determined at about week 17 and from about week 25 to and including about week 33. In some embodiments, clinical response is determined during the drug holiday, e.g., from about week 5 (after injection of the final optional dose of the adenovirus in the first treatment cycle) to and including about week 16.

In some embodiments an eligible individual for the additional treatment cycle shows clinical response after the first treatment cycle but before the second treatment cycle. In some embodiments an eligible individual shows partial clinical response. In some embodiments an eligible individual shows stable disease. In some embodiments an eligible individual shows progressive disease.

In some embodiments the treatment achieves a beneficial clinical response (e.g. partial response or stable disease) in at least one of the one or more target tumors after the first treatment cycle, but before the second treatment cycle. In some embodiments the disclosed treatment does not achieve complete response (e.g. achieves partial response or stable disease or progressive disease) in at least one of the one or more target tumors after the first treatment cycle, but achieves complete response in at least one of the one or more target tumors after the second, third, or fourth treatment cycle. In some embodiments the disclosed treatment achieves a beneficial clinical response (e.g. complete response, partial response, or stable disease) in at least one of the one or more target tumors. In some embodiments the disclosed treatment achieves a beneficial clinical response (e.g. complete response, partial response, or stable disease) in the one or more non-target tumors.

In some embodiments the treatment is effective to achieve histological clearance in at least one of the one or more target tumors receiving the adenovirus (e.g., SP-002 (ASN-002)) injection, histological clearance in at least one of the one or more non-target tumors not receiving the adenovirus (e.g., SP-002 (ASN-002)) injection, or both. In some embodiments, histological clearance is determined after the completion of a first treatment cycle. In some embodiments, histological clearance is determined after the completion of the second, third, or fourth treatment cycles. In some embodiments, histological clearance is determined after the completion each of the treatment cycles. In some embodiments, histological clearance is determined after the completion of a first treatment cycle but before the initiation of a second treatment cycle, e.g., during the drug holiday, e.g., from about week 5 to and including about week 16. In some embodiments, histological clearance is determined at about week 16, about week 17, or about week 18, e.g., at the start of or during the second treatment cycle. In some embodiments, histological clearance is determined after completion of the first and optional second treatment cycle, e.g., from about week 25 to and including about week 33, e.g., about week 25, about week 26, about week 27, about week 29, about week 29, about week 30, about week 30, about week 31, about week 32, or about week 33. In some embodiments, histological clearance is determined at about week 17 and from about week 25 to and including about week 33.

In some embodiments the treatment achieves histological clearance in at least one of the one or more target tumors. In some embodiments the treatment achieves histological clearance in at least one the one or more non-target tumors. In some embodiments the treatment achieves complete response and histological clearance in at least one of the one or more target tumors. In some embodiments the treatment further achieves complete response and histological clearance in at least one of the one or more non-target tumors.

In some embodiments the treatment achieves a reduction in the rate per year of surgically-eligible BCCs (e.g., tumors of 5 mm or greater in size).

In some embodiments the skin cancer comprises more than one target tumors.

In some embodiments, skin cancer is selected from the group consisting of basal cell carcinoma (BCC), squamous cell carcinoma, and melanoma. In certain embodiments, the skin cancer is basal cell carcinoma (BCC). In some embodiments, the basal cell carcinoma (BCC) is Basal Cell Nevus Syndrome, Gorlin syndrome, sporadic BCC, multiple BCC, nodular BCC, superficial BCC, advanced BCC, metastatic BCC or recurrent BCC. In some embodiments, the individual has BCC lesions that have recurred after radiotherapy. In some embodiments, the individual has BCC lesions that are unresectable. In some embodiments, the individual has BCC lesions in which surgical resection would result in substantial deformity. In certain embodiments, the BCC involves increasing tumor size. In certain embodiments, the BCC has BCC lesions on the central face (e.g., on the nose, eyes, mouth, chin, cheeks, forehead). In certain embodiments, the BCC has poorly defined clinical margins. In certain embodiments, the recurrent BCC is peri-neural or peri-vascular BCC (which confers higher risk of recurrence). In certain embodiments, the BCC is locally advanced basal cell carcinoma that has recurred following surgery. In certain embodiments, the BCC is in an individual in need thereof who is not a candidate for surgery and/or who is not a candidate for radiation.

In some embodiments the individual is an adult. In certain embodiments, the individual is a senior adult. In some embodiments the individual is a juvenile.

### EXAMPLES

In order that this disclosure may be more fully understood, the following Examples are set forth, It should be understood that these examples are for illustrative purposes only and are not to be construed as limiting this disclosure in any manner.

### Example 1. A Phase 2A Study to Assess the Safety and Efficacy of SP-002 (ASN-002) Combined with a Hedgehog Pathway Inhibitor in the Treatment of Multiple Low Risk Basal Cell Carcinomas in Sporadic or Basal Cell Nevus Syndrome Patients

This study is a non-randomized, parallel assigned, open label, phase 2a clinical trial. In this study up to 24 participants have been enrolled, and an additional 60 patients may be enrolled. The purpose of the study is to assess the safety and efficacy of the adenovirus SP-002 (ASN-002) in combination with vismodegib for the treatment of multiple low risk Basal Cell Carcinomas in sporadic or Basal Cell Nevus Syndrome patients. This study also evaluates SP-002 (ASN-002) and vismodegib as monotherapies.

Patients assigned to the treatment arm with vismodegib received a daily dose of 150 mg for a 4-week treatment period. Patients assigned to the treatment arm with SP-002 (ASN-002) received 3 weekly injections for 3 weeks (i.e., three injections in total). The study initially evaluated two Arms receiving 1.0×10¹¹ vp/injection, and following a safety review, implemented further Arms in an adaptive study design (low dose of 0.5×10¹¹ vp per target tumor, and high dose of 1.5×10¹¹ vp/injection). Following the first dose of SP-002 (ASN-002) (week 3), patients were monitored for 2 hours, and following subsequent doses of SP-002 (ASN-002), patients were monitored for 1 hour for signs and symptoms of an adverse reaction. Any reaction observed during this time were monitored, managed, and documented by the study site personnel, or if no reaction was observed, patients were free to return home. Patients assigned to a treatment arm with both vismodegib and SP-002 (ASN-002) completed an induction phase with vismodegib prior to SP-002 (ASN-002) treatments weekly for 2 weeks (from Day 1 to Day 14) in Cycle 1, and optionally an induction phase with vismodegib for 1 week (from Day 1 to Day 7) of Cycle 2.

Prior to the weekly administration of SP-002 (ASN-002), the investigator should review each tumor for injection site inflammatory responses. SP-002 (ASN-002) administration may be deferred by a week in any tumor with an injection site inflammatory response of Grade 3. There should be no corresponding delay to vismodegib therapy.

A study schedule and administration schedule of vismodegib and SP-002 (ASN-002) is summarized in Table 2. Day 29 injection of SP-002 (ASN-002) in Cycle 1 may be optional for a variation on the Study Design in Phase 3. Day 15 and Day 22 injections of SP-002 (ASN-002) in Cycle 2 may be optional for a variation on the Study Design in Phase 3.

TABLE 2

| Cycle | Treatment Week | Cycle Day | Vismodegib | SP-002 (ASN-002) |
|---|---|---|---|---|
| Cycle 1 | Week 1 to Week 4 | Day 1 to Day 14 | X | |
| | | (Weeks 1-2) | | |
| | | Day 15 (Week 3) | X | X |
| | | Day 16 to Day 21 | X | |
| | | (Week 3) | | |
| | | Day 22 (Week 4) | X | X |
| | | Day 23 to Day 28 | X | |
| | | (Week 4) | | |
| | Week 1 to Week 5 | Day 29 (optional) | | X |
| | | (Week 5) | | (optional) |
| Drug | Week 5 to Week 16 | | | |
| holiday | (after final optional | | | |
| | injection) | | | |
| Assessment | Week 17 | | | |
| of Clinical | | | | |
| response | | | | |
| Cycle 2 | Week 17 to Week 20 | Day 1 to Day 7 | X | |
| | | (Week 17) | | |
| | | Day 8 (Week 18) | X | X |
| | | Day 9 to Day 14 | X | |
| | | (Week 18) | | |
| | | Day 15 (Week 19) | X | X |
| | | | | (optional) |
| | | Day 16 to Day 21 | X | |
| | | (Week 19) | | |
| | | Day 22 (Week 20) | | X |
| | | | | (optional) |
| | | Day 22 to Day 28 | X | |
| | | (Week 20) | | |
| Assessment | Week 25 to Week 33 | | | |
| of clinical | | | | |
| response; | | | | |
| Excision | | | | |
| (Histological | | | | |
| analysis) | | | | |

Clinical assessment was conducted at week 17. In response to the clinical assessment, the investigator may initiate another 4 weeks of dosing with vismodegib and single SP-002 (ASN-002) injection following Cycle 2 dosing. Surgical excision is to occur between week 25 and week 33 at the investigator's discretion, and dependent on when patient completed study treatment (1 or 2 treatment cycles). Up to 10 BCCs are to be excised including 3 target tumors. Excisions can be conducted over 2 visits as per investigator's discretion. Histological response will be evaluated in all study tumors via excision between week 25 and week 33 (as per investigator's discretion).

The primary outcome of this study measures incidence of SP-002 (ASN-002) related adverse events in patients with previously untreated Nodular Basal Cell Carcinoma (nBCC). Participants will be followed up to 6 months. Incidence of adverse events will be monitored. The primary outcome of this study also measures microscopic clearance of the injected target BCC. Microscopic examinations of sample collected at weeks 25-33 after the first dose. Histological clearance (HC) will be defined as the absence of detectable evidence of BCC tumor cell nests in serial histological samples as determined by central pathology review.

The secondary outcome of this study measures microscopic clearance of the injected non-target BCC. Microscopic examinations of sample collected at weeks 25-33 after the first dose. Histological clearance (HC) will be defined as the absence of detectable evidence of BCC tumor cell nests in serial histological samples as determined by central pathology review.

### Selection Criterial for Study BCC Tumors (Target and Non-Target)

Clinical criteria for selection of study BCC tumors (target and non-target) include: primary, previously untreated tumor; 5 mm to 20 mm in diameter; clearly defined borders; absence of ulceration; not in site of prior radiation therapy; must be able to excise with primary closure; tumors on the following locations may not be study tumors: scalp, lips, within 2 cm of the open eyelid margins, within 1 cm of a scar or an area previously treated with surgical excision, breasts, or the dorsum of the hand.

Histological criteria for selection of study BCC tumors (target and non-target) include: superficial and at least 1 nodular BCC tumors (without any other mixed component except for superficial) will be eligible. Tumors that demonstrate a pattern other than standard nodular and/or superficial BCC on the biopsy specimen will be excluded. Tumors with of the following pathological features, will not be eligible: morpheaform, sclerosing, or mixed infiltrative histology; micronodular histology; basosquamous (keratinising) histology; cystic tumors; adnexal differentiation; adenoid tumors; fibroepithelioma of Pinkus; mixed histological subtypes; perineural invasion; follicular involvement; keratinising foci in the tumor. Target tumors should be at least 8 cm apart.

### Eligibility and Exclusion Criteria

A patient will be considered eligible for inclusion in this study only if all of the following criteria apply: 1) histologically confirmed previously untreated BCC (nodular and superficial), 5-20 mm in maximum diameter, per the selection criteria for study BCC tumors. A punch biopsy (refer to study procedure manual for biopsy size selection) from the thickest part of all the target tumors is required for histological confirmation of BCC and to exclude BCC non-eligible subtypes. If a patient has mix of nodular and superficial BCC tumors, at least one target tumor should be a nodular BCC; 2) Removal of <25% of the area of each biopsied tumor by initial biopsy performed 1-12 weeks before day 1. If the initial biopsy was performed>8 weeks prior to screening, the investigator may re-biopsy the tumor, provided not >25% of the area of the original tumor is removed. Non-study tumors may be resected or treated at the discretion of the Investigator prior to study entry or if they develop during the study; 3) Hedgehog pathway inhibitor treatment naïve; 4) Acceptable general health as determined by the investigator, i.e. no serious or active medical or psychiatric illness or recreational or therapeutic drug or alcohol use that, in the opinion of the Investigator, would interfere with treatment, assessment or compliance with the protocol, ability to provide informed consent, or patient safety; 5) 18 years of age or older; 6) Eastern Cooperative Oncology Group (ECOG) performance status 0, 1 or 2; 7) Screening laboratory values as follows: a. neutrophil count>1500/mm³; b. haemoglobin>9 g/dL; c. platelet count>100,000/mm3; d. prothrombin INR<1.5; e. total bilirubin<1.5× upper limit of normal (ULN), except in the case of known Gilbert's syndrome; f. aspartate transaminase (AST), alanine transaminase (ALT) or alkaline phosphatase (ALP)<2×ULN; g. creatinine<1.5× upper limit of normal (ULN); 8) Female patients who are documented infertile, postmenopausal for at least 1 year, surgically sterile or using acceptable and highly effective birth control for the duration of the study and for at least 3 months after last administration of the study treatments; 9) Male patients with female partners of child bearing potential, agreement to use two adequate contraception methods while being on vismodegib and for 3 months of completion, agreement not to donate semen for 3 months after completion of vismodegib; 10) Written informed consent prior to initiation of study-specified procedures; 11) Able and willing to comply with all study requirements, including surgical removal of tumor/tumor site at completion of study; 12) Baseline tissue sample adequate for determination of histological or other biomarkers.

The exclusion criteria for the study include: 1) known or suspected metastatic disease or other active, invasive malignancy; 2) female patients of childbearing potential who are lactating or pregnant (negative serum pregnancy test needed prior to dosing); 3) clinically active or uncontrolled skin disease or tattoos that would interfere with evaluation of the area surrounding the target tumor (e.g. eczema, unstable psoriasis, xeroderma pigmentosa); 4) known history of sensitivity to any of the ingredients in SP-002 (ASN-002) and any Hh pathway inhibitors; 5) immunocompromised (e.g. known Hepatitis B or C infection, HIV infection) or receiving or expected to receive an immune modulating agent (including immune suppressive agents, cytotoxic drugs, biological agents, immunoglobulins, interferon or other immune or cytokine-based therapies; use of inhaled or oral corticosteroids at doses higher than physiological replacement doses is an exclusion criterion); 6) has received or is expected to receive treatment with psoralen plus UVA or UVB therapy within 6 months of the screening visit; 7) any prior systemic anti-tumor therapy or local treatment for target tumors prior to first dose; 8) history of immunological disorder, severe allergic reaction, moderate or severe asthma or known history of anaphylaxis or any other serious adverse reactions to any medication; 9) any experimental or investigational agents within one month of first SP-002 (ASN-002) injection; 10) any prior exposure to TG1041, TG1042 (SP-002 (ASN-002)), any other adenoviral-based experimental agent, or any form of gene therapy within 6 months of first dose of vismodegib in the study; 11) any prior exposure to vismodegib or any other Hh inhibitor within 6 months of first dose in the study; 12) current therapy with any medications recognized to cause rhabdomyolysis or a prior history of rhabdomyolysis.

### Results

Interim data for nine patients in Arms 1 and 2 (Arm 1=single target tumor; Arm 2=multiple target tumors) with nodular BCC administered 1.0×10¹¹ vp/injection SP-002, each receiving the optional SP-002 injection at week 5, demonstrated the dosing regimen was well tolerated, with complete clinical response observed in 100% of the target tumors and a complete histological clearance rate of 85.7% per tumor (or 87.5% per patient for target tumor). Non-target tumor complete clinical response was higher for the combination regimen (23.5%) compared to the monotherapy (5%). Six of the nine patients required only one treatment cycle. Two patients who, upon assessment at week 17, did not respond to the 1″ cycle treatment with complete clinical response received a second treatment cycle, the data for which is provided in Table 3. Each target tumor (TT) and non-target (NT) tumor evaluated were biopsied at Week 33. Patient 1 in Arm 1 demonstrated complete response in the single tumor treated, but upon histological analysis, did not achieve histological response as the treatment area still contained a small amount of residual nodular BCC in the superficial dermis. Patient 2 in Arm 2 demonstrated complete clinical response and complete histological response in all target tumors (TT1, TT2, TT3) treated. In the non-target tumors (NT1, NT2, and NT3), the patient achieved partial clinical response, with residual nodular BCC extending to the mid-dermis (NT1) or superficial dermis (NT2) upon histological analysis. For NT3 histology, areas of the tumor which achieved PR showed intact epidermis and overlying dermis and subcutis, and within the superficial dermis an increase in thin walled vascular channels in keeping with a (benign) haemangioma, showing no evidence of dysplasia or malignancy, which is in keeping with a finding of no residual disease (NRD).

TABLE 3

| | | | | | Lesion size (mm) | | | | | | | | | |
|---|---|---|---|---|---|---|---|---|---|---|---|---|---|---|
| | | | | | 1^{st} cycle | | | | DH | 2^{nd} cycle | | Excision | | |
| Patient | Arm | Tumor | | Site | Day 0 | Wk 3 | Wk 4 | Wk 5 | Wk 7 | Wk 17 | Wk 18 | Wk 33 | Clinical | Histological |
| 1 | 1 | TT1 | | RH trapezius | 7 | 4 | 0 | 0 | 4 | 1 | 3 | 0 | CR | RD |
| 2 | 2 | TT1 | | Chest | 7 | 6 | NA | NA | NA | 1 | 1 | 0 | CR | NRD |
| | | TT2 | | RH upper back | 8 | 5 | NA | NA | NA | 1 | 1 | 0 | CR | NRD |
| | | TT3 | | RH flank | 7 | 7 | NA | NA | NA | 1 | 1 | 0 | CR | NRD |
| | | | NT1 | RH shoulder | 7 | 7 | 9 | 7 | 5 | 6 | 7 | 6 | PR | RD |
| | | | NT2 | RH scapula | 8 | 11 | 6 | 5 | 5 | 5 | 6 | 2 | PR | RD |
| | | | NT3 | RH lower back | 6 | 6 | 5 | 5 | 4 | 6 | 5 | 4 | PR | NRD |

NA = tumor lesion size was indeterminate; DH = Drug holiday from last day of treatment on Week 5 to Week 16; Day 0 = lesion size measured before treatment; CR = complete response; PR = partial response; RD = residual disease; NRD = no residual disease.

### OTHER EMBODIMENTS

This application refers to various issued patent, published patent applications, journal articles, and other publications, each of which is incorporated herein by reference.

The foregoing has been described of certain non-limiting embodiments of the present disclosure. Those of ordinary skill in the art will appreciate that various changes and modifications to this description may be made without departing from the spirit or scope of the present disclosure, as defined in the following claims.

1. A method of treating skin cancer in an individual in need thereof, comprising:
a) administering to the individual a first treatment cycle of vismodegib and a replication-deficient type 5 adenovirus for expression of interferon gamma, wherein the first treatment cycle comprises oral daily administration of 150 mg vismodegib to the individual for 4 weeks of the first treatment cycle and intralesional injection of the adenovirus to one or more target tumors in a dose range from about 0.5×10¹¹ vp to about 1.5×10¹¹ vp per tumor per dosing day on one day of week 3, one day of week 4, and optionally one day of week 5, of the first treatment cycle;
b) after administering the final dose of the first treatment cycle, providing a drug holiday; and then
c) administering to the individual a second treatment cycle of vismodegib and the adenovirus, wherein the second treatment cycle comprises oral daily administration of 150 mg of vismodegib to the individual for 4 weeks of the second treatment cycle and intralesional injection of the adenovirus to the one or more target tumors in a dose range from about 0.5×10¹¹ vp to about 1.5×10¹¹ vp per tumor per dosing day to the individual one day of week 2, optionally one day of week 3, and optionally one day of week 4, of the second treatment cycle.

2. The method of claim 1,
wherein the first treatment cycle comprises oral daily administration of 150 mg vismodegib to the individual on each one of days 1 to 28 of the first treatment cycle and intralesional injection of the adenovirus to one or more target tumors in a dose range from about 0.5×10¹¹ vp to about 1.5×10¹¹ vp per tumor per dosing day to the individual on day 15, day 22, and optionally day 29 of the first treatment cycle; and
wherein the second treatment cycle comprises oral daily administration of 150 mg of vismodegib to the individual on each one of days 1 to 28 of the second treatment cycle and intralesional injection of the adenovirus to the one or more target tumors in a dose range from about 0.5×10¹¹ vp to about 1.5×10¹¹ vp per tumor per dosing day to the individual on day 8, optionally on day 15, and optionally on day 22, of the second treatment cycle.

3. The method of claim 1, wherein the intralesional injection of the adenovirus to one or more target tumors in a dose range from about 0.5×10¹¹ vp to about 1.5×10¹¹ vp per tumor per dosing day to the individual is given on one day of week 5 or day 29 of the first treatment cycle.

4. The method of claim 1, wherein the treatment achieves a beneficial clinical response in at least one of the one or more target tumors after the first treatment cycle, but before the second treatment cycle, wherein the beneficial clinical response is partial response or stable disease.

5. The method of claim 4, wherein the beneficial clinical response is partial response.

6. The method of claim 4, wherein the beneficial clinical response is stable disease.

7. The method of claim 1, wherein the treatment does not achieve complete response in at least one of the one or more target tumors after the first treatment cycle, but achieves complete response in at least one of the one or more target tumors after the second treatment cycle.

8. The method of claim 7, wherein the treatment achieves partial response after the first treatment cycle.

9. The method of claim 7, wherein the treatment achieves stable disease after the first treatment cycle.

10. The method of claim 1, wherein the treatment achieves clinical response in the one or more target tumors, wherein the clinical response is partial response or stable disease.

11. The method of claim 1, wherein the treatment achieves clinical response in at least one of the one or more non-target tumors, wherein the clinical response is partial response or stable disease.

12. The method of claim 1, wherein the treatment achieves histological clearance in at least one of the one or more target tumors.

13. The method of claim 1, wherein the treatment achieves histological clearance in at least one of the one or more non-target tumors.

14. The method of claim 1, wherein the treatment achieves complete response and histological clearance in at least one of the one or more target tumors.

15. The method of claim 14, wherein the treatment further achieves complete response and histological clearance in at least one of the one or more non-target tumors.

16. The method of claim 1, wherein the drug holiday is at least 8 weeks.

17. The method of claim 1, wherein the drug holiday is from about week 5, after the last dose of the first treatment cycle, to and including week 16.

18. The method of claim 17, wherein the second treatment cycle is initiated from about week 17 to about week 20 after the initiation of the first treatment cycle.

19. The method of claim 18, wherein the treatment achieves clinical response in at least one or more target tumors from about week 25 to and including about week 33.

20. The method of claim 1, wherein the treatment achieves histological clearance in at least one of the one or more target tumors receiving the adenovirus injection, histological clearance in at least one of the one or more non-target tumors not receiving the adenovirus injection, or both, from about week 25 to and including about week 33.

21. The method of claim 1, wherein the treatment achieves a reduction in the rate per year of surgically-eligible BCCs.

22. The method of claim 1, wherein the dose of the adenovirus intralesional injection is about 1.0×10¹¹ vp per tumor per dosing day.

23. The method of claim 1, wherein the dose of the adenovirus intralesional injection is about 1.5×10¹¹ vp per tumor per dosing day.

24. The method of claim 1, wherein the dose of adenovirus intralesional injection is about 0.5×10¹¹ vp per tumor per dosing day.

25. The method of claim 1, wherein a volume of the adenovirus intralesional injection is up to about 0.5 mL for tumors with a diameter of no more than 10 mm.

26. The method of claim 1, wherein a volume of the adenovirus intralesional injection is up to about 1.0 mL for tumors with a diameter of more than 10 mm.

27. The method of claim 1, wherein the skin cancer is selected from the group consisting of basal cell carcinoma (BCC), squamous cell carcinoma, and melanoma.

28. The method of claim 27, wherein skin cancer is basal cell carcinoma (BCC).

29. The method of claim 28, wherein the BCC is Basal Cell Nevus Syndrome, Gorlin syndrome, sporadic BCC, multiple BCC, nodular BCC, superficial BCC, advanced BCC, metastatic BCC, and/or recurrent BCC; and/or the individual has BCC lesions that have recurred after radiotherapy, BCC lesions that are unresectable or surgical resection would result in substantial deformity, BCC involving increasing tumor size, BCC lesions on the central face, BCC with poorly defined clinical margins, recurrent BCC such as peri-neural and peri-vascular BCC, locally advanced BCC that has recurred following surgery and/or in individuals in need thereof who are not candidates for surgery and/or who are not candidates for radiation, or any combination thereof.

30. The method of claim 1, wherein the skin cancer comprises more than one target tumor.

31. The method of claim 1, wherein the individual is an adult.
